(19) United States
(12) Patent Application Publication
(10) Pub. No.: US 2007/0161985 A1
(43) Pub. Date: Jul. 12, 2007

(54) Screws configured to engage bones, and methods of attaching implants to skeletal regions

(75) Inventors: John J. Demakas, Spokane, WA (US); Brent W. Johnston, Spokane, WA (US)

Correspondence Address: WELLS ST. JOHN P.S., 601 W. FIRST AVENUE, SUITE 1300, SPOKANE, WA, 99201, US

(73) Assignee: Kentomia, LLC .

(21) Appl. No.: 11/295,181

(22) Filed: Dec. 5, 2005

(51) Int. Cl. A61F 2/30

(52) U.S. Cl. 606/61

(57) ABSTRACT

The invention includes screws configured to directly engage bones, with such screws including pores configured to receive bone structure grown from the bone to enhance union of the screw with the bone. The screws can be, for example, pedicle screws, and in some aspects can have bone-growth-stimulating material and/or bone cement within the pores. The invention also includes methods of attaching implants to skeletal regions. Such methods can include screwing a porous screw into the bone, allowing bone structure to grow from the bone into the porous screw, and subsequently fastening an implant to the porous screw.

### TECHNICAL FIELD

The invention pertains to screws configured to directly engage bones, and in some aspects pertains to pedicle screws. The invention also pertains to methods of attaching implants to skeletal regions.

### BACKGROUND OF THE INVENTION

There are numerous implant constructions configured for attachment to living bones, including, for example, screws, plates, cages and rods. The implants can be provided for numerous reasons including, for example, as temporary support to immobilize a skeletal region during healing in response to injury (for instance, screws, rods and/or plates utilized to immobilize a fractured bone during healing of the fracture), as permanent support to replace a skeletal segment (for example, a knee or hip replacement), or as permanent support to provide additional support beyond that offered by a skeletal region compromised by injury, disease, aging or genetic defect (for example, spinal implant constructions provided for additional support beyond that offered by a deteriorated spine).

A difficulty in attaching implant constructions to skeletal regions is that numerous conditions and diseases can lead to softened or weakened bone structures to which it is difficult to achieve robust union. For instance, osteoporosis increases bone porosity, which leads to softened bone structures. Implant constructions can frequently be screwed to osteoporotic bones in a problem-free manner. However, the screws holding the implant constructions to the bones can subsequently loosen from the bones through the normal forces exerted on the screws and implant constructions during ordinary day-to-day activities, or even can be pulled out of the bones if large forces occur.

Similar difficulties to those confronted with softened or weakened bone structures can also occur with normal, healthy bone structures.

In light of the problems confronted in obtaining and maintaining robust union of screws with bones, it is desired to develop new methods for adhering screws to living bones.

An exemplary prior art procedure of attaching an implant construction to a skeletal region is described with reference to FIGS. 1-3 to assist the reader in understanding a general process for attachment of implant constructions. It is to be understood, however, that the invention claimed herein can have applications beyond the specific exemplary application of FIGS. 1-3.

Referring to FIG. 1, such shows an assembly 10 comprising a spine 12 and a pair of implant constructions 20 and 30.

The spine comprises a series of vertebrae 14, 16 and 18 separated by disks 15 and 17.

The implant construction 20 comprises a rod 22 held between a pair of support structures 24 and 26; and the implant construction 30 comprises a rod 32 held between a pair of support structures 34 and 36. The rods 22 and 32 would traditionally be relatively rigid metal bars (such as, for example, titanium bars), but it is becoming increasingly common to utilize somewhat flexible materials (such as, for example, polymeric materials) for the rods to provide increased mobility. The support structures 24, 26, 34 and 36 contain screws inserted into the pedicles of the vertebra. Such screws have heads configured to enable retention of the rods. The support structures also comprise plugs inserted into the heads of the screws to lock the rods into the screws, as described in more detail below with reference to FIGS. 2 and 3.

A spinal segment is typically defined as a disc and the pair of vertebra on opposing sides of the disc. Thus, the implant constructions 20 and 30 can each be considered to comprise a pair of pedicle screws on opposing sides of a spinal segment, and a rod joining the pedicle screws to one another.

FIG. 2 shows a cross-section through vertebra 18, and through support structures 24 and 34 of the constructions 20 and 30. The cross-section of FIG. 2 shows various anatomical features of vertebra 18, including the vertebral body 40, spinal canal 42 (through which the spinal nerve (not shown) passes), and pedicles 44 and 46. The cross-section of FIG. 2 also shows that support structures 24 and 34 comprise pedicle screws 50 and 60, respectively, which extend through pedicles 44 and 46, and into the vertebral body 40.

The pedicle screws 50 and 60 have heads 52 and 62, respectively. Such heads have channels 54 and 64 extending therein. The channels are configured to receive rods 22 and 32, and are further configured to receive plugs (or caps) 56 and 66 which retain the rods within the channels. The particular shown screws have threads within the channels. The threads within the channels receive threads of the plugs so that the plugs can be threadedly engaged within the channels to retain the rods. However, as will be recognized by persons of ordinary skill in the art, there are numerous other structural designs for pedicle screw heads which can be utilized for retaining rods to the pedicle screws. Also, persons of ordinary skill in the art will recognize that pedicle screws can be utilized for retaining other implant structures besides rods.

FIG. 3 shows a disassembled structure 70 comprising a pedicle screw 72 and a cap 74. The screw 72 is identical to the screws 50 and 60 discussed above the reference to FIG. 2, and the cap 74 is identical to the caps 56 and 66. The disassembled structure of FIG. 3 shows that the cap is configured to threadedly engage within the channel in the head of screw 72.

The implant constructions of FIGS. 1 and 2 are typically provided in a multi-step surgical procedure. First, an incision is made to expose the region where the implant construction is to be placed, and specifically to expose the vertebral pedicles. The pedicle screws are then screwed into the pedicles utilizing an appropriate tool. The pedicle screws can be self-tapping, or, if not, tapped holes can be formed in the pedicles prior to inserting the screws into the pedicles. The pedicle screws will typically have one or more tool engagement slots provided within the heads of the screws. Such tool engagement slots can, for example, correspond to slots configured to receive any appropriate tool for screwing the screws into the pedicles, including, for example, a Phillips screwdriver or other cross-slotted screwdriver, a straight-slotted screwdriver, an Allen wrench, a Torx wrench, etc.

In the next step of the procedure, the rods are provided within the channel regions at the heads of the pedicle screws, and the caps (for instance, 54 or 64 of FIG. 2) are threaded into the channel regions to lock the rods in place. The caps will typically have one or more tool engagement slots provided therein to enable an appropriate tool to be utilized for screwing the caps into place. Such tool engagement slots can, for example, correspond to slots configured to receive any appropriate tool for screwing the screws into the pedicles, including, for example, a Phillips screwdriver or other cross-slotted screwdriver, a straight-slotted screwdriver, an Allen wrench (Allen wrench type slots are shown in the caps in FIG. 1), a Torx wrench, etc.

In the next step of the procedure, the incision is closed.

The spinal implant constructions and procedures discussed above are illustrative of a few of the many types of implant constructions and procedures. Numerous types of screws can be utilized for attachment to various skeletal regions. The invention described and claimed below can have application to any screw utilized for permanent attachment to a skeletal region. However, pedicle screws can be particularly problematic for utilization in patients (both people and animals) suffering from deteriorative bone disease, and the invention described below can, in some aspects, be of particular usefulness for utilization with pedicle screws.

### SUMMARY OF THE INVENTION

In one aspect, the invention includes a screw configured to directly engage a living bone. The screw comprises a shaft that is at least partially threaded, and at least one pore extending into the shaft and configured to receive bone structure grown from the bone.

In one aspect, the invention includes a pedicle screw having one or more cavities extending therein, and having a bone-growth-stimulating material within at least one of said one or more cavities.

In one aspect, the invention includes a method of attaching an implant construction to a skeletal region. The method includes the following steps in the following listed sequence. Initially, a porous screw is screwed into a bone, with term “porous” indicating that the screw is porous relative to osteoblasts and growing bone. A period of time is then allowed to pass for bone structure to grow from the bone into one or more pores of the porous screw. Finally, the implant construction is fastened to the screw.

### BRIEF DESCRIPTION OF THE DRAWINGS

Preferred embodiments of the invention are described below with reference to the following accompanying drawings.

FIG. 1 is a diagrammatic view of a prior art assembly comprising a spine and implant constructions attached to the spine.

FIG. 2 is a cross-section along the line 2-2 of FIG. 1.

FIG. 3 is a diagrammatic side view of a disassembled prior art pedicle screw assembly.

FIG. 4 is a diagrammatic side view of a pedicle screw in accordance with an exemplary aspect of the present invention.

FIG. 5 is a cross-sectional side view of the pedicle screw of FIG. 4, and specifically is a view along the line 5-5 of FIG. 4.

FIG. 6 is a diagrammatic cross-sectional side view of another embodiment of a screw formed in accordance with an aspect of the present invention.

FIG. 7 is a diagrammatic cross-sectional side view of a skeletal region having an exemplary screw of the present invention retained therein.

FIG. 8 is a flow-chart diagram describing an exemplary methodological aspect of the present invention.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

The invention includes new methods and structures for fastening implant constructions to skeletal regions. The methods and structures can be utilized in veterinary applications for treating animals, or can be utilized for treating humans. In particular aspects, the invention includes incorporation of pores within screws that are engaged into bone, with the pores being configured so that bone structure grown from the bone extends into the pores to improve the union of the screw with the bone. The bone structure growth into the pores can be enhanced by providing one or more bone-growth-stimulating compositions within the pores and/or by providing bone cement within the pores.

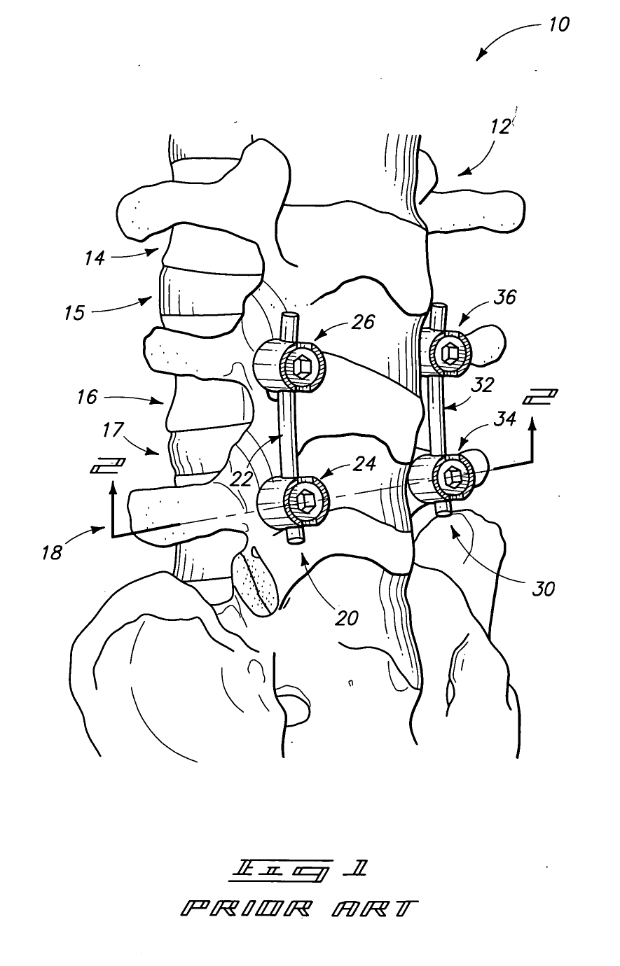

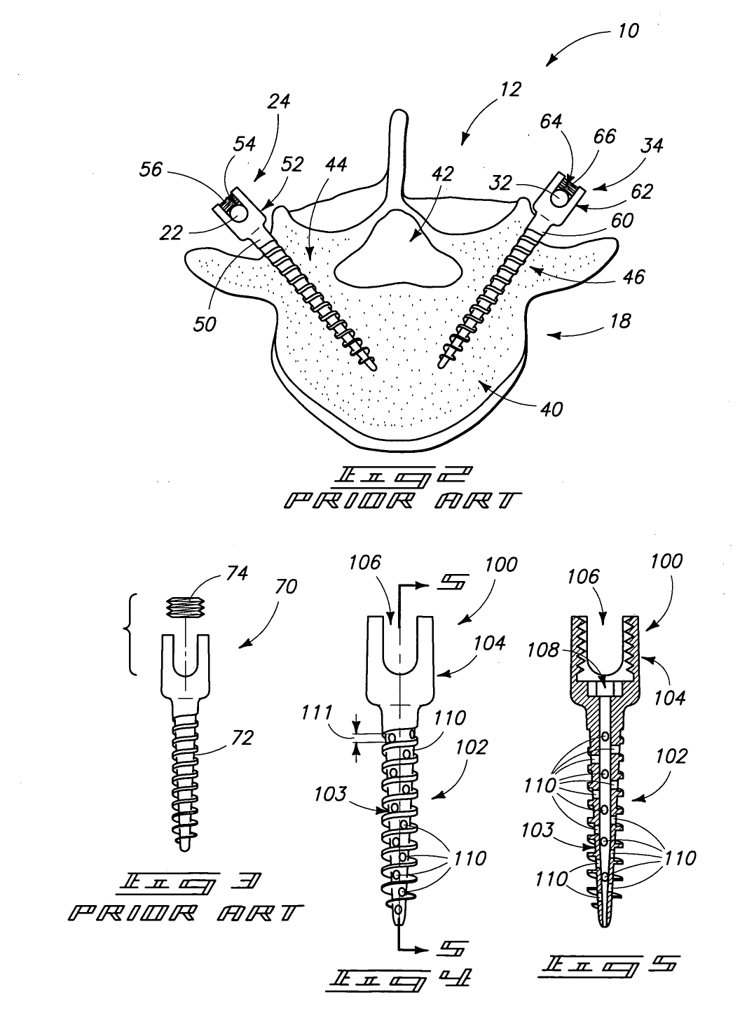

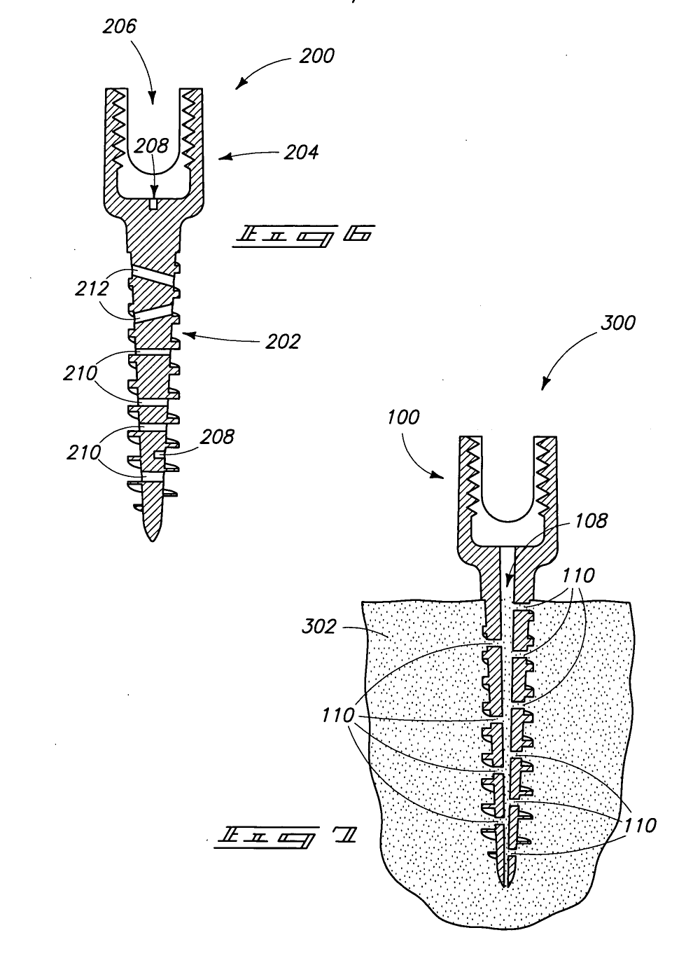

FIGS. 4 and 5 show an exemplary screw 100 illustrating an aspect of the present invention. The screw is a pedicle screw similar to the prior art screws 50, 60 and 70 discussed above with reference to FIGS. 1-3. Accordingly, screw 100 comprises a threaded shaft 102 and a head 104 joined to the shaft. The shaft 102 is shown to be fully threaded, but it is to be understood that the shaft could also be only partially threaded in some applications.

The head 104 has a channel 106 extending therein. Such channel is threaded, as is apparent from the cross-sectional view of FIG. 5. The channel is configured so that a cap (or plug) can be utilized for retaining a rod within the channel. The screw 100 of FIGS. 4 and 5 can be a pedicle screw having a length of at least about 30 mm and a widest diameter of at least about 5 mm.

The screw 100 of FIGS. 4 and 5 differs from the prior art screws of FIGS. 1-3, in that screw 100 comprises a longitudinally-extending opening (also referred to herein as a cannula) 108 within the shaft, and a plurality of pores 110 (only some of which are labeled) extending through the shaft and to the opening 108. In some aspects, the shaft 102 can be considered to comprise a lateral sidewall 103, and the pores can be considered to extend through such lateral sidewall to the longitudinally-elongated opening 108. The pores and opening are configured to enable bone growth to extend into the screw 100. The lateral sidewall 103 can be considered an outer sidewall surface of the shaft, with at least a portion of such outer sidewall surface being threaded.

Although screw 100 differs from prior art screws, persons of ordinary skill in the art will recognize that a tool can be readily configured for inserting screw 100 into a bone.

The size of the longitudinally-elongated opening, size of the pores, and number of pores can vary depending on the intended application of screw 100. In some applications (discussed below with reference to FIG. 6), the longitudinally-elongated opening can be omitted. In some aspects of such applications, at least some of the pores can extend entirely through the screw (i.e., entirely from one lateral side of the screw to the opposing lateral side of the screw).

In applications in which the longitudinally-elongated opening is provided, the longitudinally-elongated opening can have any suitable length relative to the length of the shaft. In the shown application, the longitudinally-elongated opening is about the same length as the length of the shaft, but in other applications the longitudinally-elongated opening can be substantially shorter than the overall length of the shaft. Typically, however, if the longitudinally-elongated opening is provided within the shaft, the longitudinally-elongated opening will be at least about one third of the length of the shaft. The longitudinally-elongated opening can function to enable bone growth to extend within the screw, and in some applications (discussed below) the longitudinally-elongated opening can also be utilized for provision of bone-growth-stimulating compositions and/or bone cement. Alternatively, or additionally, the longitudinally-elongated opening can be utilized as a reservoir for retaining bone-growth-stimulating compositions and/or bone cement. In some aspects of the invention, it can be preferred that the longitudinally-elongated opening extend to the channel in the head, as shown, to enable bone-growth-stimulating compositions and/or bone cement to be injected into the longitudinally-elongated opening after the screw is at least partially inserted into a bone.

Regardless of whether or not a longitudinally-elongated opening is provided within the screw 100, there will be at least one pore (or cavity) extending into or through the wall of the shaft, and specifically through the bottom (i.e., tip) of the shaft and/or through a sidewall of the shaft. In the shown aspect of the invention, a pore extends through the bottom of the shaft, and several pores extend through the sidewall of the shaft. If the shaft is only partially threaded, one or more pores can extend into non-threaded portions of the shaft in addition to, or alternatively to, having one or more pores extending into threaded portions of the shaft.

Pores 110 can have any suitable size for enabling sufficient bone growth to occur within the pores to assist in retaining the screw to a bone. The shown pores are approximately circular along a lateral cross-section, with an exemplary pore having a cross-sectional diameter 111 of, for example, from about 0.1 mm to about 3 mm. The pores can extend through the sidewall 103 at any suitable angle. In some aspects, the pores will extend substantially orthogonally to a normal (i.e., longitudinal) axis of the screw, and in other applications at least some of the pores will extend at an angle which is not substantially orthogonal to the normal axis of the screw.

Although the screw of FIG. 5 is shown as a pedicle screw, it is to be understood that the screw can alternatively be another type of screw suitable for engaging bone. For instance, the screw can be a cervical screw, or a screw suitable for engaging regions other than the spine and cervix, including, for example, screws suitable for retaining hip implants, knee implants or shoulder implants; screws suitable for being utilized alone to retain bone fragments; or screws suitable for retaining various plates, cages and rods; or any other screws utilized for reconstruction, repair and/or support of skeletal regions. The screws can be based on prior art screws with the modification that screws of the present invention have pores extending therein suitable for receiving bone structure grown from a living bone to enhance union of screws of the present invention with the bone. Accordingly, screws of the present invention can have numerous shapes and sizes, as will be recognized by persons of ordinary skill in the art.

FIG. 6 shows a screw 200 illustrating another aspect of the present invention. Screw 200 is a pedicle screw similar to the screw 100 discussed above with reference to FIG. 5. Screw 200 comprises a threaded shaft 202 and a head 204 joined to the shaft. The screw 200 further comprises a threaded channel 206 extending into the head. Channel 206 has a slot 208 therein for receiving a tool which can be utilized for screwing the screw 200 into a bone. Such slot can be part of receptacle suitable for receiving, for example, a Phillips screwdriver or other cross-slotted screwdriver, a straight-slotted screwdriver, an Allen wrench, a Torx wrench, etc.

Screw 200 differs from the screw 100 of FIGS. 4 and 5 in that screw 200 lacks a longitudinally-elongated opening analogous to the opening 108. However, screw 200 still comprises pores 208, 210 and 212 analogous to the pores 110 associated with the screw 100 of FIGS. 4 and 5, with such pores being laterally-elongated openings in the embodiment of FIG. 6. The pores 208, 210 and 212 are configured for receiving bone structure grown into the pores. In the shown aspect, some of the pores extend entirely through the screw (specifically, pores 210 and 212) while one of the pores only extends partially into the screw (specifically, pore 208). Also, some of the pores are shown extending along approximately horizontal axes relative to a vertical axis defined by a normal axis of the screw (specifically, pores 210) while some of the pores are shown extending along axes tipped relative to such horizontal axes (specifically, pores 212).

The screws of FIGS. 4-6 can be formed of any suitable composition or combination of compositions. In some aspects, the screws will consist essentially of, or consist of metal; and in particular aspects the screws can consist essentially of, or consist of titanium.

Regardless of whether a porous screw is configured with a longitudinally-extending opening of the type shown in FIGS. 4 and 5, or without such longitudinally-elongated opening as shown in FIG. 6, bone-growth-stimulating material and/or various cements and bone adhering materials can be provided in one or more of the pores. For instance bone-growth-stimulating material can be provided to enhance growth of bone into the pores and/or polymethyl-methacrylate (PMMA) (a form of bone cement) can be provided within the pores to enhance adhesion to bone. If the longitudinally-elongated opening is present, the bone-growth-stimulating material and/or PMMA can be provided by injection of the bone-growth-stimulating material and/or PMMA through the longitudinally-elongated opening and into the pores joined to the opening before, after, and/or during screwing of the screw into bone. If the longitudinally-elongated opening is not present, the bone-growth-stimulating material and/or PMMA will typically be provided in the pores prior to screwing of the screw into the bone. Also, even if the longitudinally-elongated opening is present, the bone-growth-stimulating material and/or PMMA can be provided within the pores but not within the longitudinally-elongated opening, or vice versa. Further, if the longitudinally-elongated opening is present but some of the pores do not join with the opening, bone-growth-stimulating material and/or PMMA can be provided within the pores that do not join with the opening prior to screwing of the screw into the bone.

The bone-growth-stimulating material can comprise any composition or combination of compositions which stimulate bone growth. For instance, the bone-growth-stimulating material can comprise one or both of fibronectin and hydroxyapatite. Additionally, or alternatively, the bone-growth-stimulating material can comprise one or more bone morphogenetic proteins (bmp's) such as, for example, bmp2 and/or bmp7; and/or other osteo-inductive conductors.

In some aspects, at least portions of the outer sidewall surfaces of the screw shafts (and particularly at least portions of the threaded surfaces of the shafts) are coated with one or both of fibronectin and hydroxyapatite to enhance union of the screws to bone. Such coating can be utilized in addition to the provision of bone-growth-stimulating material and/or bone cement in the pores and/or cannula of porous screws.

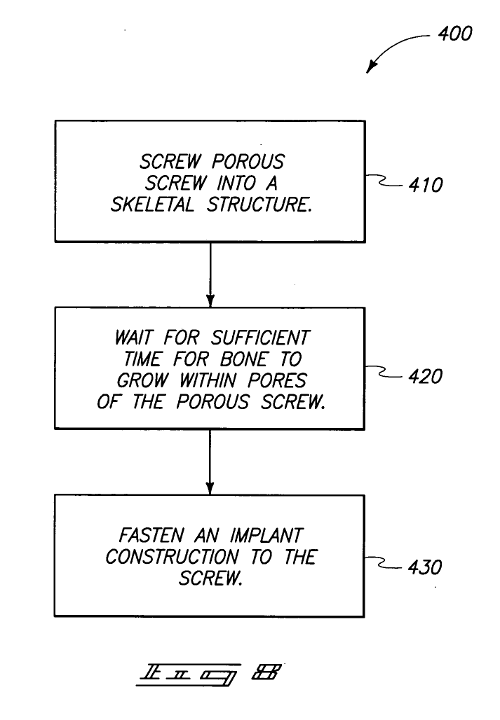

FIG. 7 shows an assembly 300 comprising the screw 100 (described above with reference to FIGS. 4 and 5) embedded in a bone 302. Structure, or matrix, of the bone is shown extending into the pores 110 of the screw, and also into the longitudinally-elongated opening 108. The bone structure within the pores and longitudinally-elongated opening enhances union of the screw with the bone. Such can alleviate prior art problems of screw loosening and screw pullout that were discussed above in the “background” section of this disclosure. Advantages of having bone growing into pores associated with a screw can occur in numerous applications, but can be particularly significant for patients suffering from bone-weakening ailments such as, for example, osteopenia or osteoporosis.

The shown screw 100 is a pedicle screw, and in the diagram of FIG. 7 bone has grown into the pores of the screw prior to assembly of the spine-stabilizing implant that is ultimately to be retained by the screw (specifically, prior to provision of rods and plugs of the type described with reference to FIG. 1). This can be a preferred aspect of the invention. Specifically, a porous screw can be fastened to a bone, and then left attached to the bone for a period of time sufficient to have bone growth extend into pores of the screw prior to attachment of an implant construction to the screw. This can enable the screw to become tightly joined with the bone through the growth of bone structure into the pores associated with the screw prior to providing stresses on the screw associated with an attached implant construction.

In the case of pedicle screws, for example, significant stresses can be applied to the screws once that rods are tightly joined to the screws. Such stresses can cause the screws to pull out of the pedicles if the stresses occur before a strong union of the screws with the pedicles has been achieved. Accordingly, it can be advantageous to wait until bone matrix material has grown into the pores of the pedicle screws (and in some aspects adhered to a surface of the screw) before tightly attaching the rods to the pedicle screws. Similar considerations can occur with screws other than pedicle screws in other applications in which the screws are utilized to support an implant construction, including, for example, applications in which the screws hold cages, plates, shafts and/or rods.

FIG. 8 is a flow-chart diagram 400 describing a procedure which can be utilized to fasten an implant construction to a bone in accordance with an exemplary aspect of the present invention.

At an initial step 410, at least one porous screw is screwed into a skeletal structure (i.e., into one or more bones). Such can occur in a first surgical procedure.

The wound formed in the first surgical procedure can then be covered and/or closed, and then the second step 420 can proceed where a sufficient time is allowed to pass for bone to grow within pores of the porous screw (or screws) to achieve a desired union of the screw (or screws) with the skeletal structure. The period of time can be any suitable time, such as, for example, at least about seven days, at least about two weeks (14 days), at least about four weeks, or even longer.

Subsequently, step 430 proceeds where an implant construction is fastened to the screw (or screws). Step 430 will typically be a second surgical procedure separate from the first procedure.

In a particular aspect, the procedure of FIG. 8 can be considered to be designed to allow stabilization of the porous screws through growth of bone onto the surface of the screws and into the perforations in the screw shafts; and can be further considered to be a two stage (or specifically, two surgery) operation. A first surgery would place the screws into the bone, (such as, for example, pedicles) and then close the wound. After sufficient time for new bone ingrowth to occur, (which can be documented by imaging, and which presently would typically be about from about 4 weeks to about 12 weeks), the patient is subjected to the second surgical procedure for the definitive operation and or the attachment of rods, plates or other devices to the screws for stabilization of a skeletal region.

In some aspects, the procedure of FIG. 8 is utilized to provide a pair of pedicle screws into vertebral pedicles on opposing sides of a spinal segment at the initial step 410 (the screws can be referred to as first and second pedicle screws, and the vertebral pedicles can be referred to as first and second vertebral pedicles), and to then provide a rod linking the pedicle screws to one another at step 430.

Although it can be preferred that bone growth form matrix material within a porous screw prior to attachment of additional implant structures to the screw in some aspects the invention; it is to be understood that the invention also includes aspects in which a porous screw is attached to a skeletal structure, and implant structures are attached to the screw prior to growth of bone matrix material into pores of the screw. The eventual growth of bone matrix material into the pores of the screw will ultimately enhance union of the screw with the bone. Accordingly, in some aspects of the invention, screws of the present invention can be utilized in place of the conventional screws now utilized without further modification of present procedures.

In some aspects, bone cement, (for example, PMMA) can be utilized with porous screws for securing the screws to a skeletal region. The bone cement can be a primary agent for securing the screws to the skeletal region, or can be an aid utilized in addition to another primary agent for securing the screws. For instance, the primary agent utilized for securing the screws can ultimately be bone grown within the pores, and the bone cement can aid in securing the screws as the bone grows into the pores. Regardless of whether the bone cement is the primary agent for securing the screws or is more of a secondary agent, the screws can be considered to be at least partially secured to the skeletal region with the bone cement.

If bone cement is utilized with the porous screws, a surgical procedure can comprise the two surgical stages of FIG. 8, or alternatively can comprise a single surgical stage in which bone is exposed, the screws are screwed into the bone and utilized to retain rods, plates or other devices, and the wound is then closed. In some aspects, the porous screws can comprise a cannula down the center (such as the cannula 108 of FIGS. 4 and 5), and pores leading through the shaft to the cannula (exemplary pores are shown in FIGS. 4 and 5). In such aspects, the screws can be screwed at least partially into bone, and then bone cement can be injected through the cannula and into at least some of the pores, and utilized to at least partially secure the screws to the bone.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. A screw configured to directly engage a bone, the screw comprising:
a shaft that is at least partially threaded;
at least one pore extending into the shaft and configured to receive bone structure grown from the bone to enhance union of the screw with the bone.

2. The screw of claim 1 being a cervical screw.

3. The screw of claim 1 being a pedicle screw.

4. The pedicle screw of claim 3 having a length of the shaft, and having a longitudinally-elongated opening within the shaft which extends along at least about one-third of the length of the shaft; the pedicle screw further having a lateral sidewall along the shaft, and having at least one pore extending through the lateral sidewall and to the longitudinally-elongated opening.

5. The pedicle screw of claim 4 comprising at least two pores extending through the lateral sidewall and to the longitudinally-elongated opening.

6. The pedicle screw of claim 4 wherein the length is at least about 30 mm, and further having a diameter of at least about 5 mm.

7. A pedicle screw having one or more pores extending therein, and having one or both of bone cement and bone-growth-stimulating material within at least one of said one or more pores.

8. The pedicle screw of claim 7 comprising the bone cement within said at least one of said one or more pores, and wherein the bone cement comprises PMMA.

9. The pedicle screw of claim 7 comprising bone-growth-stimulating material within said at least one of said one or more pores.

10. The pedicle screw of claim 9 wherein the bone-growth-stimulating material comprises fibronectin and hydroxyapatite.

11. The pedicle screw of claim 10 wherein the bone-growth-stimulating material further comprises one or more bone morphogenetic proteins.

12. The pedicle screw of claim 7 having a threaded shaft with a length, and having a longitudinally-elongated opening extending within the shaft and along at least some of the length, and wherein at least one of said one or more pores extends to the longitudinally-elongated opening.

13. The pedicle screw of claim 12 wherein at least one of the pores having the one or both of PMMA and bone-growth-stimulating material therein also extends to the longitudinally-elongated opening, and wherein the one or both of PMMA and bone-growth-stimulating material is within the longitudinally-elongated opening.

14. The pedicle screw of claim 12 having a head attached to the shaft, with said head having a channel extending therein; and wherein the longitudinally-elongated opening extends into the shaft from the channel in the head.

15. A method of attaching an implant construction to a skeletal structure, comprising:
screwing one or more porous screws into the skeletal structure;
providing bone cement within at least one pore of at least one of the porous screws;
at least partially securing said at least one of the porous screws to the skeletal structure with the bone cement; and
fastening the implant construction to the one or more porous screws.

16. The method of claim 15 wherein the one or more screws have threaded shafts which engage the skeletal structure upon screwing the screws into the skeletal structure, and further comprising coating at least a portion of the threaded shaft of at least one of the screws with one or both of fibronectin and hydroxyapatite prior to screwing said at least one of the screws into the skeletal structure.

17. The method of claim 16 said at least a portion of the threaded shaft is coated with a mixture of fibronectin and hydroxyapatite.

18. The method of claim 15 wherein the providing the bone cement occurs prior to screwing the at least one screw into the skeletal structure.

19. The method of claim 15 wherein the providing the bone cement occurs after screwing the at least one screw at least partially into the skeletal structure.

20. The method of claim 15 wherein said one or more porous screws is a plurality of porous screws, and wherein the bone cement is provided within at least one pore of all of the porous screws.

21. The method of claim 15 wherein the bone cement comprises PMMA.

22. The method of claim 15 wherein said at least one of the one or more porous screws has a longitudinally-elongated channel contained therein and at least one pore extending to the channel, and wherein at least some of the bone cement is provided through the channel and into the at least one pore after at least partially screwing the at least one porous screw into the skeletal structure.

23. The method of claim 15 wherein the skeletal region includes two vertebral pedicles on opposing sides of a spinal segment, wherein the porous screws include a first pedicle screw extending into one of said vertebral pedicles and a second pedicle screw extending into the other of said vertebral pedicles, wherein both the first and second porous screws are at least partially secured with bone cement, and wherein the implant construction includes a rod extending from the first pedicle screw to the second pedicle screw.

24. A method of attaching an implant construction to a skeletal region, comprising the following steps in the following sequence:
screwing one or more porous screws into a skeletal structure;
waiting a period of time for bone structure to grow from the skeletal structure into one or more pores of the one or more porous screws; and
fastening the implant construction to the one or more porous screws.

25. The method of claim 24 wherein the period of time is at least about seven days.

26. The method of claim 24 wherein the period of time is at least about two weeks.

27. The method of claim 24 wherein the one or more screws have threaded shafts which engage the skeletal structure upon screwing the screws into the skeletal structure, and further comprising coating at least a portion of the threaded shaft of at least one of the screws with one or both of fibronectin and hydroxyapatite prior to screwing said at least one of the screws into the skeletal structure.

28. The method of claim 27 said at least a portion of the threaded shaft is coated with a mixture of fibronectin and hydroxyapatite.

29. The method of claim 24 further comprising providing bone cement within one or more of the pores.

30. The method of claim 29 wherein the bone cement includes PMMA.

31. The method of claim 24 further comprising providing a bone-growth-stimulating material within one or more of the pores.

32. The method of claim 31 wherein at least some of the bone-growth-stimulating material is provided within said one or more of the pores prior to the screwing of the one or more porous screws into the skeletal structure.

33. The method of claim 31 wherein an entirety of the bone-growth-stimulating material provided within at least one of the one or more porous screws is provided prior to the screwing of said at least one porous screw into the skeletal structure.

34. The method of claim 31 wherein at least one of the one or more porous screws has a longitudinally-elongated channel contained therein, and wherein at least some of the bone-growth-stimulating material is provided within said at least one porous screw after at least partially screwing the at least one porous screw into the skeletal structure.

35. The method of claim 31 wherein the bone-growth-stimulating material comprises fibronectin and hydroxyapatite.

36. The pedicle screw of claim 35 wherein the bone-growth-stimulating material further comprises one or more bone morphogenetic proteins.

37. The method of claim 24 wherein the skeletal region includes two vertebral pedicles on opposing sides of a spinal segment, wherein the porous screws include a first pedicle screw extending into one of said vertebral pedicles and a second pedicle screw extending into the other of said vertebral pedicles, and wherein the implant construction includes a rod extending from the first pedicle screw to the second pedicle screw.
